(19) United States
(12) Patent Application Publication
(10) Pub. No.: US 2002/0146741 A1
(43) Pub. Date: Oct. 10, 2002

(54) Processes for receptpor screening

(76) Inventors: Cale M. Halbleib, Madison, WI (US); Kerry M. Ervin, Middleton, WI (US); Mohammed S. Shekhani, Madison, WI (US); Robert G. Lowery, Brooklyn, WI (US); William J. Checovich, Madison, WI (US); Thomas J. Burke, Madison, WI (US)

Correspondence Address: Mark K. Johnson, PO Box 510644, New Berlin, WI, 53151-0644, US

(21) Appl. No.: 09/918,589

(22) Filed: Jul. 30, 2001

Related U.S. Application Data
(60) Provisional application No. 60/221,621, filed on Jul. 28, 2000.

(51) Int. Cl. G01N 33/53

(52) U.S. Cl. 435/7.1

(57) ABSTRACT

A method is provided for measuring competitive binding activity of molecules to nuclear receptors comprising mixing a fluorescence-emitting compound that specifically binds to steroid hormone receptors in a solution wherein the fluorescence-emitting compound is in concentration below the dissociation constant and the steroid hormone receptors are at or above the dissociation constant using fluorescence polarization.

This application is related to provisional application serial No. 60/221,621 which filed on Jul. 28, 2000.

### BACKGROUND

The steroid receptor superfamily is a class of ligand activated transcription factors that are involved in regulation of almost all aspects of cell growth and metabolism. Thus it is not surprising that from a pharmaceutical perspective they are considered key targets for therapeutic intervention for a wide range of diseases and metabolic disorders; these include breast and prostate cancers, osteoporosis, inflammation, and diabetes. To enable these drug discovery opportunities, simple, robust assay methods that can be used to assess large diverse chemical collections for identification of novel steroid receptor ligands are needed. In addition, the ability to assess manmade and naturally occurring chemicals found in soil and water for potential disruption of endocrine functions has become an important priority for environmental agencies and industrial manufacturing groups.

Identification of improved drug molecules for modulating SHR function requires methods to rapidly screen large chemical libraries for functional interaction with the receptors. Previous to our lab's efforts in this area, investigators relied on impure preparations of rodent receptors and cumbersome radioactive assay methods to assess binding of test compounds to steroid receptors. Screening the hundreds of thousands of compounds typical of pharmaceutical compound collections was not practical using these methods. To overcome this technical impasse, our lab has developed a series of molecular SHR ligand binding assays that operate in a “mix and read” format: test compounds are added to a mixture of recombinant SHR protein and a fluorescent tracer compound and the relative binding affinity of the test compounds are ascertained by a single instrument reading without any additional manipulations. The assay principle is fluorescence polarization: the ability of the test compound to displace the fluorescent tracer from the SHR binding pocket is proportional to the degree to which the fluorescence of the tracer becomes depolarized. Pharmaceutical scientists can now test tens of thousands of compounds for SHR binding in a single day using any one of a number of commercially available fluorescence plate readers typically used for automated high throughput screening. Thus, this technology should accelerate the development of more selective steroid receptor modulators to address a wide range of diseases and metabolic disorders.

SHRs are Key Targets for Therapeutic Intervention

The steroid hormone receptors (SHRs) are a subclass of the nuclear receptor superfamily, which includes a total of 46 members in humans. The SHR subclass is comprised of the estrogen receptors (ER), the androgen receptor (AR), the glucocorticoid receptor (GR), the progesterone receptors (PR), and the mineralcorticoid receptor (MR). These proteins mediate ligand-induced transactivation of genes responsible for cellular differentiation, reproduction, and metabolism. Upon binding hormone, SHRs in association with their cognate DNA response elements, undergo a conformational change leading to binding with coactivator proteins which direct the recruitment of cellular transcriptional machinery. Because of their involvement with cell growth and differentiation, SHRs participating in cellular pathways gone awry have been implicated in numerous disease states.

Estrogen receptor is a 66 kDa member of the nuclear receptor superfamily of ligand activated transcription factors. It is involved in control of growth, differentiation and function in mammary tumors and in diverse target tissues including reproductive, skeletal, and cardiovascular tissues.

Androgen receptor is a 99 kDa protein that binds testosterone and dihydrotestosterone (DHT) and is involved in sexual maturation, spermatogenesis, and bone metabolism. Recent studies have indicated that AR is expressed in prostate tumors of all stages and mutated forms of AR are present in late-stage metastatic tumors. While the detection of prostate abnormalities has greatly improved with prostate-specific antigen (PSA) tests, treatment of prostate cancer by androgen ablation therapy via surgery or administration of anti-hormones often works for a limited time, after which the tumor becomes resistant. This resistance has been attributed to AR gene amplification and to mutation of the AR leading to altered ligand specificity; one such mutated AR will be included in the Phase II studies. Since prostate cancer has become the most commonly diagnosed cancer in the male population, research to find novel anti-androgens for resistant tumors has become a high priority. In addition to its role in reproductive cancers, AR has recently been found in osteoclast cells where it is believed to play a role in bone deposition and prevention of osteoporosis in men. The osteoclast AR demonstrated response to a different stereoisomer of alpha-DHT than AR in the prostate indicating that it may be possible to develop selective modulators for AR as well as ER. Specific coactivator proteins have been identified for AR, such as androgen-receptor associated 70 protein (ARA70) and the recently cloned ARA54 and ARA55. These coactivators are expressed in prostate tumors and interact with mutated AR forms to varying degrees. A novel co-regulator for AR named ARIP3 has been found in human testes and represents a tissue-specific regulator, adding another degree of complexity to AR transactivation.

Progesterone receptor exists as two forms referred to as A and B with molecular masses of 94 kD and 114 kD, respectively. PR-A lacks N-terminal sequences compared to PR-B and results from an alternative translation start site. PR is expressed in the reproductive system, mammary tissue, and brain and has been implicated in breast cancer, endometriosis, and uterine fibroids. Like estrogen, progesterone is believed to play a role in regulation of bone mass, and hence development of bone specific agonists may be useful for prevention of osteoporosis. PR-A and PR-B can form heterodimers and have distinct ligand specificities and transactivation capabilities. PR-B functions as a transactivator whereas PR-A functions as a dominant repressor of PR-B and even down-regulates other SHRs including ER (28,30-33). Various ratios of PR-A:PR-B have been found in breast cancer tissues.

Glucocorticoid Receptor (GR) is a 94 kDa ligand-activated intracellular transcriptional regulator that is a member of the nuclear receptor superfamily. GR is the mediator of glucocorticoid action that regulates the metabolism of carbohydrates, proteins, and fats, suppresses the immune/inflammatory responses, activates the Central Nervous System, regulates cardiovascular function, and affects basal and stress-related homeostasis. Glucocorticoid therapies are used in the treatment of asthma, chronic arthritis, inflammatory bowel disease, lymphoma, leukemia, hyperglycemia, renal and pulmonary conditions, multiple sclerosis, and headaches. GR is the only SHR that absolutely requires association with heat shock and other proteins to exhibit ligand binding activity, thus it is not possible to use a highly purified from of the receptor for in vitro assays.

Assays for SHR Ligand Binding

Detection and characterization of SHR agonists and antagonists can be done at three levels of complexity: whole organism (bioassays), cell based assays, and molecular assays. In this discussion, the whole organism and cell based approaches will be classified as in vivo assays and contrasted with the molecular assay, an in vitro method. Though each approach is useful for certain aspects of drug development and toxicological testing, the molecular assay based on direct detection of the interaction of ligands with the receptor is the most useful for screening large, diverse chemical libraries in an automated, high throughput format.

The classic method for measuring steroidogenic effects is based on the ability of a compound to affect the development of secondary sexual characteristics in vivo. For example, test compounds must be fed or injected into mice over a period of three days, followed by surgical removal and weighing of the sexual organs. These assays measure specific biological outcomes of ligand binding to SHRs, and are still used by investigators studying SHR effects in the reproductive system. However, the time and intensive labor required for the in vivo methods preclude their use for screening large numbers of compounds.

A number of cell based assays have been developed to circumvent the difficulty of the bioassays; examples of these are induction of proliferation in the breast carcinoma cell line MCF-7 for ER, or the prostate tumor cell line LNCaP for AR, or induction of reporter gene expression from a hormone-responsive promoter transfected into yeast or mammalian cells. All of these measure the biological consequences of ligand binding within a specific cell and promoter context, thus a panel of such assays would provide a relatively broad functional characterization of ligands. However the cell proliferation and reporter gene assays require extensive manipulations of live cells and response times of several hours or even days.

Molecular ligand binding assays are faster, more precise, and less labor intensive than animal or cell-based assays because they are done in vitro with isolated components. These advantages allow accurate measurements of receptor and other assay components in an automated format and eliminate signal variability inherent in any vivo assay. In addition, in vitro assays do not require any of the cell growth and maintenance steps required for in vivo assays, and thus are much easier to format for high throughput screening.

More importantly, using the initial molecular event (ligand binding) in steroid hormone receptor signal transduction as a primary screen supports a rational approach of starting with a broad screen and applying increasing levels of selectivity. Such a funneled approach insures that compounds of potential interest are not eliminated by a primary screen that is too selective.

Ligand Binding Assays: Several methods are used for SHR ligand binding assays. All are competitive assays, in which the test compound displaces a receptor-bound probe molecule—generally radiolabelled hormone. As explained above, there are compelling reasons for using the initial molecular event in ER signal transduction to develop high throughput screening assays. However, the current methods for detecting this event suffer from a number of important shortcomings, generally caused by the use of relatively crude receptor preparations, the requirement for a separation step to remove free ligand, and the use of radioisotopes. Unfortunately, these shortcomings prevent the widespread adaptation of current SHR ligand binding assays to HTS formats.

To develop ligand binding assays more suited to an HTS format, investigators have begun using various types of scintillation proximity assays, or flash plate assays, in which the receptor and the scintillant are bound to a solid phase such as a bead or a plate to ensure that only the receptor bound labeled hormone—and not the excess free ligand—is close enough to excite the scintillant. This approach eliminates the need to separate free from bound ligand, but still relies upon the use of radioisotopes and immobilization of the receptor, which could cause disadvantageous conformational changes. In addition, antibodies often are used to immobilize the receptor, thus introducing another source of assay variability from possible interference of test compounds with Ab-antigen interactions, and possible degradation of the antibodies.

### BRIEF DESCRIPTION OF THE DRAWINGS

FIG. 1A and B are graphs showing the binding of tritiated steroids to recombinant human GR and PR-LBD. The binding affinity of baculovirus expressed GR for dexamethasone was determined by a steroid-binding assay. GR (8 nM) was incubated with varying concentrations of [³H] dexamethasone for 2 hrs at 4° C. Unbound [³H] Dex was removed with dextran coated charcoal. Tracer in the soluble GR fraction was counted in a liquid scintillation counter. The K_{d} for [³H] dexamethasone was found to be 4.5 nM. The PR assay was done similarly using dextran coated charcoal to remove the free tritiated steroid, but in this case a constant concentration of ³H-progesterone (4 nM) was titrated with recombinant PR-LBD. The observed K_{d} was 0.2 nM.

FIG. 2A and B are graphs showing saturable binding of recombinant PR-LBD and GR to their respective fluorescent steroid ligands. Serial dilutions of GR and PR-LBD were prepared and added to black plastic multiwell plates containing and 1 nM fluorescent glucocorticoid and 2 nM fluorescent progesterone, respectively. Plates were incubated 1 hour at room temperature. The background fluorescence for each GR and PR-LBD concentration was subtracted for each well. For the GR/GS1 complex, the apparent K_{d} was 0.8±0.1 nM and for PR-LBD/PL1 complex it was K_{d}=2.5 nM. The final incubation conditions for the GR binding assay were 10 mM potassium phosphate (pH 7.4), 10 mM Na₂MO₄, 0.1 mM EDTA, 2% DMSO, 5 mM DTT and 100 μM stabilizing peptide (LPYEGSLLLKLLRAPVEEV) and 1 nM fluorescent glucocorticoid. Final conditions for the PR-LBD binding assay were 100 nM KPO₄ (pH 7.4), 10% glycerol, 100 μg/ml bovine gamma globulin, 0.02% NaN₃, 2 mM DTT, and 2 mM fluorescent progesterone.

FIG. 3A and B are graphs showing FP-based competition assays with recombinant GR (A) and PR-LBD (B). Serial dilutions of test steroids were prepared. Fluorescein conjugates of dexamethasone and progesterone were added sequentially to each well in combination with GR and PR-LBD, respectively. Final incubation conditions were similar to those described in FIG. 3. The final concentration of GR used was 4 nM, and for PR-LBD it was 8 nM. After a 1-hour incubation at room temperature, the fluorescence polarization of each well was read, using wells lacking fluor as blanks. IC₅₀ values and RBAs for each steroid were determined from the average of 10 plate reads. Note that the RBA's reflect literature values.

FIG. 4A and B show saturable ligand binding and competition binding curves for PR with a tetramethyl-rhodamine (TMR) conjugate of progesterone. This data demonstrates the use of fluorescent molecules other than fluorescein as conjugates to steroids for use as FP assay probes. The TMR-progesterone conjugate was developed in a manner similar to that described above for Fluorescein-steroid conjugates. A: 2 nM TMR-progesterone was titrated with the GST-PRLBD fusion protein in multiwell plates using conditions described in FIG. 3 and fluorescence polarization readings were taken using a Tecan Polarion. B: The preformed TMR-progesterone/GST-PRLBD complex was tirated with estradiol or progesterone and allowed to come to equilibrium for several hours at 4° C. prior to FP readings.

### SUMMARY

Use of Fluorescence Polarization for Ligand Binding Assays

To fully explain how fluorescence polarization can be used to overcome the shortcomings of existing methods for SHR ligand identification and characterization, it is helpful to briefly summarize the principles of the technique. Fluorescence polarization (FP) is used to study molecular interactions by monitoring changes in the apparent size of fluorescently-labeled or inherently fluorescent molecules. When a small fluorescent molecule (probe) is excited with plane polarized light, the emitted light is largely depolarized because the molecule rotates rapidly in solution during the fluorescence event (the time between excitation and emission). However, if the fluorescent probe is bound to a much larger receptor, thereby increasing its effective molecular volume, its rotation is slowed sufficiently to emit light in the same plane in which it was excited. The bound and free states of the fluorescent molecule each have an intrinsic polarization value, a high value for the bound state and a low value for the free state. In a population of molecules, the measured polarization is a weighted sum of the two values, thus providing a direct measure of the fraction of the fluorescent probe molecule which is bound. The method can be used for direct measurement of probe binding in response to effectors or for competitive binding assays, where the probe molecule is displaced by added test compounds. Data from FP assays are analyzed graphically in a manner similar to that used for radioactive ligand binding assays. The use of FP for SHR competitive ligand binding assays is shown graphically below.

[image]

The magnitude of the FP signal is proportional to the apparent size of the fluorescent assay probe, thus assay probes (fluorescent steroids) have a high polarization when they are bound to the receptor and a low polarization after they are displaced from the receptor by a test compound.

FP offers several key advantages over other technologies for development of SHR ligand binding and coactivator interaction assays that can be adapted to HTS formats. (Multiwell FP instruments are readily available from three different companies.) Specifically, FP is:

Nonradioactive—By employing fluorescent probes, FP eliminates radiation handling, disposal and costs for regulatory compliance.

Homogenous—FP is a homogenous assay format: it does not require separation of free from bound probe. The fluorescent probe molecule and the test compound are added to the receptor to form a mixture which is allowed to reach equilibrium and then measured with no further manipulations. This eliminates the need to attach the receptor to a solid phase and/or any centrifugation, filtration, and wash steps, thereby preventing distortion of receptor conformation and leaching of loosely bound ligands. These factors make the technology considerably less cumbersome than existing methods, and thus much easier to format for HTS.

Able to measure true equilibrium binding—By eliminating the need for receptor immobilization and other manipulations described above, FP allows measurement of true equilibrium binding in solution. This allows accurate estimations of binding affinity, detection of low affinity ligands, and easier incorporation into a multiwell, HTS format.

Flexible—Performing assays with FP provides much more flexibility for changing reagent concentrations and testing additional variables. Because the assay does not rely on secondary binding reactions to immobilize the receptor or excess free ligand, there are fewer interdependent variables. This greatly simplifies assay optimization and design of more efficient high throughput screens because it allows more freedom in changing internal assay components, and requires fewer controls for the addition of exogenous agents. In addition, the homogenous nature of the assay allows samples to be analyzed, treated, and reanalyzed. For instance, binding data can be obtained under basal conditions, followed by a) changes in exogenous agents such as temperature or test compound concentration and b) a second measurement after the system reaches a new equilibrium. This provides additional flexibility in the type and amount of data that can be extracted from a single screen, a critical advantage when working with limited amounts of chemicals in proprietary compound collections.

Real Time Technology—Using FP, the approach to equilibrium for each binding reaction can be monitored by repeated measurements, allowing direct measurement of binding kinetics in a high throughput format.

Expression and Purification of Recombinant Steroid Hormone Receptors

It is not practical to use FP for binding assays with poorly enriched receptor fractions. Because there are no separation steps in FP, a sufficient amount of receptor must be present to allow binding by at least 50% of the fluorescent probe molecule prior to the addition of test compound. Otherwise, the unbound probe molecule decreases the starting polarization value and limits the dynamic range of the assay. To avoid this, relatively high concentrations of receptor are required, which generally are not attainable in crude preparations from native sources. In addition, because pharmaceutical drug screening programs preferentially target the human receptors, obtaining suitable quantities from natural sources is not practical. For these reasons, recombinant, purified receptors are the most useful reagents for use in an FP assay.

It is clear from the scientific literature that SHRs are particularly difficult to produce in large quantities. Some of the approaches used in the past to overcome these difficulties have included addition of ligand to the culture media in order to stabilize the receptor, and expression of smaller domains of the receptors. However, neither of these approaches is ideal for production of protein for drug screening assays; full length, unliganded receptors would be optimal for this purpose. (It is very difficult to remove bound SHR ligands because of their slow dissociation rate.) Our experience and the scientific literature suggests that BaV is the best expression system for producing large quantities of full length SHRs. In order to obtain high yields of active receptor, it is useful to systematically optimize all of the parameters that are likely to have an impact on BaV mediated expression in insect cells, including:

Multiplicity of infection and cell harvest time post-infection. We have found that an MOI between 1.0 and 5.0 is generally the most productive, and that optimal harvest times can vary from 36 to more than 72 hours post-infection.

Use of different type of host cells. “In house” strains of Sf9, Sf21 and T.Ni should be tested, but in addition, the same strains from other sources should be tested. The reason for this is that the characteristics of insect cell lines can change after many passages.

Optimization of media. Media composition can have a profound impact on expression levels in insect cells. Several commercial sources of insect cell media, both complete medias, and those that require the addition of fetal calf serum should be tested. Different sources of serum should also be tested, as these can affect expression levels as well.

Optimization of expression vectors. The nature of the DNA construct used in BaV expression is often overlooked because of the ready availability of convenient vectors and high rate of success with this system. However there are several vector properties that can have an impact on expression, including the strength of the promoter and the amount and composition of 5′ untranslated sequence. The polyhedrin promoter is by far the most commonly used for heterologous expression in insect cells, however the extremely rapid rate of transcription from this promoter may not be optimal for expression of proteins where proper folding is a problem. Thus, weaker promoters should be assessed, such as the p10 promoter to try to increase the level of soluble SHR expression. In addition, the amount of viral DNA in the 5′ untranslated region of vectors can have an impact on expression of downstream genes.

Other expression systems can also be used for SHR expression. Yeast has been used to express several nuclear receptors. Another possibility is the expression of ligand binding domains (LBD) in E. coli, most likely as an N-terminal fusion to increase solubility. Although full length protein is optimal for drug screening purposes, the LBDs of several nuclear receptors have been shown to exhibit ligand binding properties essentially identical to the full length receptors. N-terminal domains used as fusions to LBDs include glutathione S-transferase (GST), thioredoxin (TRX), and maltose binding protein (MBP). Several SHRs and other nuclear receptors have been expressed in this way and subsequently purified and crystallized. In the case of the AR-LBD even a TRX-LBD fusion protein was largely insoluble, and it was necessary to denature and refold the protein.

To purify recombinant SHRs, techniques described in the literature or developed at our lab for ERs and VDR are useful. These include using ion exchange, size exclusion, metal chelate, affinity chromatography directed against fusion domains, and hydrophobic interaction chromatography. The SHRs are very hydrophobic and tend to aggregate, thus a primary challenge is maintaining protein solubility and activity during the purification and perfoming the purification without ligand or other stabilizing reagents that could interfere with hormone receptor binding, such as most detergents. Methods to stabilize the unliganded receptor include addition of agents that prevent aggregation such as urea, KCl or glycerol and inclusion of protease inhibitors.

Applications

We are developing the FP-based competitive ligand binding assays for steroid hormone receptors primarily for drug screening purposes as in vitro assays using isolated proteins, however other applications are applicable:

The assays can be used to identify ligands for steroid hormone receptors for other purposes than drug development, such as identification of previously unknown endogenous ligands.

The approach can also be applied to whole cells which would allow detection of ligands interacting with steroid hormone receptors directly in vivo.

If background fluorescence and light scattering are reduced sufficiently, the assays have diagnostic applications for quantifying steroid hormone receptor levels in tissue samples.

The assays can be used to monitor interaction of steroid hormone receptors with other factors such as proteins or DNA that bind to the receptor in a ligand dependent fashion.

### DETAILED DESCRIPTION

We have synthesized a series of fluorophore-steroid conjugates and identified one fluorescein-estradiol conjugate that was optimal for development of fluorescence polarization (FP)-based competitive ligand binding assays for estrogen receptors (ER). The assay measures the difference in fluorescence polarization of the fluorescent ligand when it is bound to a steroid receptor and when it is displaced by a competing test ligand. We have now extended the FP based assay methods to GR and PR by developing suitable fluorescent ligands for these receptors and optimizing their use in competitive HTS binding assays. Human androgen, glucocorticoid, and progesterone receptors (AR, GR, PR) have been produced and purified, and validated for high affinity steroid binding. Fluorescent steroid ligands that bind to the glucocorticoid and progesterone have been identified and used to develop FP assays for high throughput drug screening; efforts to synthesize a high affinity fluorescent AR ligand are ongoing.

Production of the recombinant SHRs: Recombinant human steroid hormone receptors including androgen, glucocorticoid, and progesterone receptors (AR, GR, PR) have been produced and purified, and validated for high affinity steroid binding. GR was produced as a full length protein in insect cells and partially purified using ion exchange chromatography; further purification was not pursued because it removes endogenous proteins required for ligand binding activity. A fragment of PR encompassing the ligand binding domain was produced with an N-terminal glutathione transferase fusion domain. AR was also produced as a ligand binding domain, in this case fused to thioredoxin. The AR and PR LBD fusion proteins were expressed in E. coli and highly purified using affinity resins directed against the fusion domain. It was necessary to denature and refold the TRX-ARLBD protein in order to obtain receptor that bound hormone with high affinity.

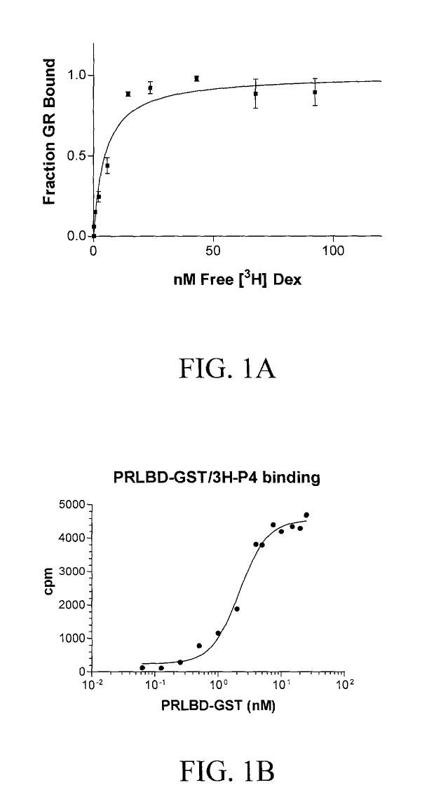

FIGS. 1A and B show that the recombinant human GR and PR-LBD used for development of FP-based competitive ligand binding assays both bind their respective steroid ligands with high affinity. High affinity steroid binding is an indication that the recombinant proteins are properly folded in their native conformation. This provides confidence that the ligand binding profiles determined in vitro will be predictive of those that occur in vivo—a necessary prerequisite for a biochemical drug screening assay.

Synthesis of fluorescent steroid conjugates. The directed combinatorial approach used for synthesis of a high affinity fluorescein-estradiol conjugate was also used to develop fluorescent GR and PR ligands and is being applied to AR as well: different combinations of powerful fluors and high affinity ligands were assembled using different linkage chemistries and tested for binding to the cognate receptor. Table 1 below shows summarizes some of the chemical components used for GR, PR and AR.

TABLE 1
The core components of the directed combinatorial approach for
identification of F-steroid assay probes for GR, AR and PR.

| Parent Steroid Ligands | Linkers | Fluorophores |
|---|---|---|
| 5□-androstan derivatives | carboxymethyloxime | Fluoresceinamine |
| 4-androsten derivatives | hemisuccinate | and 4,6-dichloro- |
| Approx. 20 compounds | | triazinyl-amino- |
| derivatized @ | | fluorescein (DTAF) |
| positions: | | are the primary |
| 1,3,6,7,11,15,17,18,19 | | fluors to be used. |
| 4-pregnen derivatives | carboxymethylether | In addition to |
| Approx. 10 compounds | acetate | fluorescein, |
| derivatized @ positions | | conjugatable |
| 3,6,7,1117,19, 20,21 | | derivatives of |
| | | BODIPY dyes, |
| | | Texas Red, |
| | | Tetramethyl |
| | | rhodamine and |
| | | Alexa dyes may |
| | | also be used |
| Dexamethasone derivatives | | |
| Approx 3 compounds | | |
| derivatized at position 21 | | |

The following systematic, iterative approach was used to produce fluorescent ligands for GR, PR and AR.

1 Conjugate fluorescein or tetramethyl rhodamine to several positions on the parent compound.

2 Purify the conjugates by TLC and quickly measure binding to the purified receptor and check for competition with unlabeled steroid.

3 If the conjugate demonstrates competitive binding, measure the dissociation constant.

4 If the conjugate has tight binding (<20 nM Kd), purify it by HPLC, collect peaks, and measure binding of the highly pure conjugates to the purified receptor.

5 If the polarization shift is <200 mP, conjugate fluorescein at the same position on the steroid but with different (generally shorter) linkages.

6 Measure binding and competition as described above.

7 If the affinity needs to be tighter, other steroids with tighter affinity can be labeled at the same ring position. For example, the 5a-androstans generally bind more tightly than the 4-androstens and would be good candidates for tighter binding if testosterone derivatives did not bind tightly enough.

High affinity, specific binding of a fluorescent steroids to GR and PR: In order to be useful for drug screening applications, the fluorescent steroids used for the assay must bind their cognate receptors specifically and with high affinity. In other words, conjugation to a fluorescent molecule must not completely disrupt the molecular interactions that allow the steroids to reside in the receptor ligand binding pocket, and must not prevent displacement by other known receptor ligands. These criteria assure that the screening assay will aid in identification of physiologically relevant ligands with the potential for meaningful modulation of SHR activity in vivo.

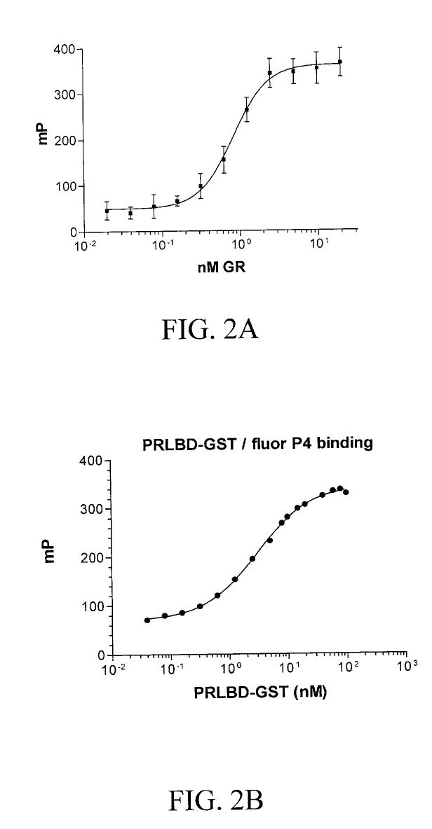

In order to test the affinity and specificity of the fluorescent steroids, they were tested in equilibrium binding experiments and in competition experiments with their cognate receptors. The equilibrium binding experiments showed that the binding of the fluorescent glucocorticoid and the fluorescent progesterone to GR and PR-LBD respectively was saturable, which is an indication of specific binding at a single site (FIG. 2). In addition, both fluorescent conjugates bound with high affinity; a K_{d} of 0.8±0.1 nM was observed for the GR/fluorescent glucocorticoid complex and a K_{d} of 2.5 nM for the PR-LBD/fluorescent progesterone complex.

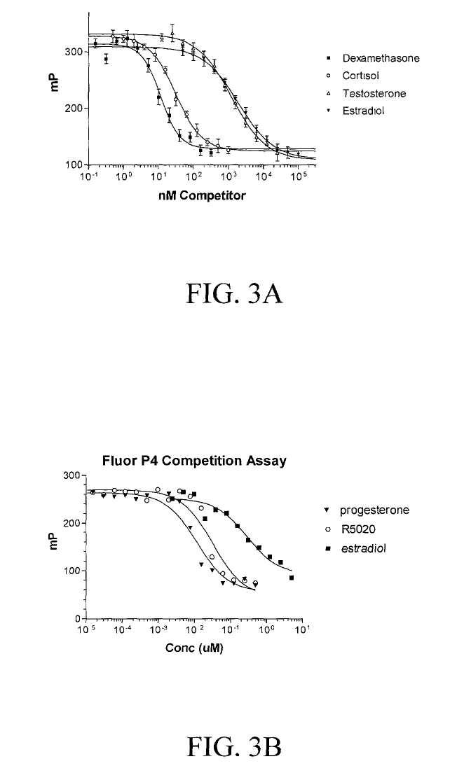

The other criteria used for validating a fluorescent steroid conjugate is the ability of known receptor ligands to compete with the fluor for binding to the receptor. This indicates that the fluorescently labeled steroid is interacting specifically with the ligand binding pocket of the receptor. FIG. 3 shows competition binding curves for the fluorescein-dexamethasone and fluorescein-progesterone conjugates with recombinant GR and PR-LBD respectively. The relative binding affinities (RBAs) of compounds determined in this assay was similar to those literature values determined using the standard radioligand binding assay described in FIG. 1. This comparison with RBAs determined with standard methodology is summarized for GR in Table 2.

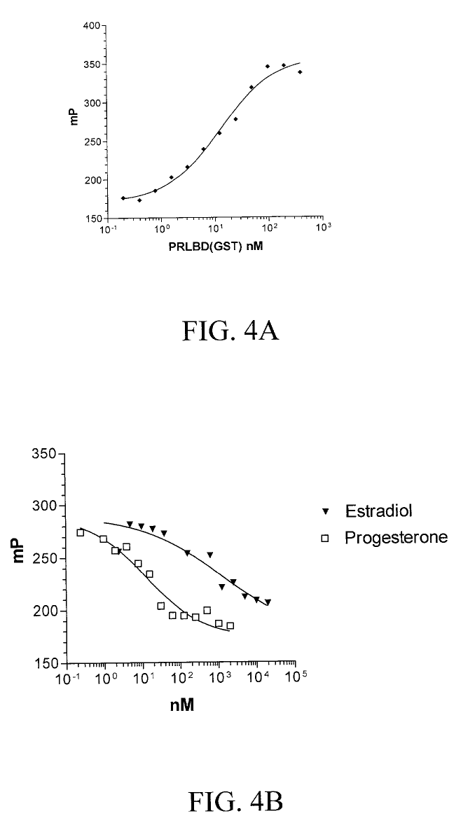

TABLE 2
GR ligand affinities determined by FP-based competitive
ligand binding assay described in FIG. 4.

| | | | Literature |
|---|---|---|---|
| Ligand | Kᵢ (nM) | RBA | Value |
| Dexamethasone | 0.74 | 100 | 100 |
| Triamcinolone acetonide | 0.90 | 82 | 100 |
| Cortisol | 3.1 | 24 | 33 |
| Fluocinonide | 4.4 | 17 | |
| 17-β-Estradiol | 620 | 0.12 | |
| Flunisolide | 1.0 | 73 | 100 |
| Amcinonide | 1.9 | 40 | |
| Betamethasone | 0.95 | 78 | |
| Halcinonide | 0.73 | 101 | |
| Prednisone | 150 | 0.50 | |
| Budesonide | 0.61 | 120 | 600 |
| Beclomethasone Monopropionate | 0.29 | 250 | 1300 |
| Beclomethasone Dipropionate | 4.6 | 16 | 50 |

In addition to fluorescein conjugates, we also have prepared conjugates of teramethyl-rhodamine to progesterone and dexamethasone. These fluorescent molecules were also demonstrated to be effective for FP-based competitive binding assays as is shown fro PR in FIG. 4.

The foregoing is considered as illustrative only of the principles of the invention. Further, since numerous modifications and changes will readily occur to those skilled in the art, it is not desired to limit the invention to the exact construction and operation shown and described. Therefore, all suitable modifications and equivalents fall within the scope of the invention.

We claim:

1. A method for measuring competitive binding activity of molecules to nuclear receptors, comprising:
a) mixing a fluorescence-emitting compound that specifically binds to steroid hormone receptors in a solution wherein the fluorescence-emitting compound is in concentration below the dissociation constant and the steroid hormone receptors are at or above the dissociation constant.
b) measuring the binding by fluorescence polarization of the solution from step a);
c) incubating the solution of step a) with at least one molecule that may compete with the compound for binding interaction with the steroid hormone receptors;
d) measuring the fluorescence polarization of the solution during step c); and,comparing the binding fluorescence polarization measurements of step b) with step d) to quantify any competitive interaction.

2. The process of claim 1 wherein the nuclear receptors include a receptor selected from the group consisting of PPAR, SXR, FXR, LXR, RXR, RAR, and TR.

3. The process of claim 1 wherein the nuclear receptor is activated by ligands other than steroids.

4. The process of claim 1 wherein the nuclear receptor is selected from the group consisting of ER, PR, and GR.

5. The process of claim 1 wherein the nuclear receptor is selected from the group consisting of MR, AR, and ERR.

6. The process of claim 1 wherein the nuclear receptor is selected from the group consisting of nuclear receptor proteins produced in bacteria or eukaryotic cells.

7. The process of claim 1 wherein the nuclear receptor is selected from the group consisting of receptors produced without the addition of ligands to a culture medium.

8. The process of claim 1 wherein the nuclear receptor is selected from the group consisting of ligand binding domains alone or fused to domains such as GST, MBP, and TRX.

9. The process of claim 1 wherein the nuclear receptor is selected from the group consisting of full length receptors, ligand binding domains and ligand binding domains fused to GST, MBP, and TRX.

10. The process of claim 1 wherein the fluorescence emitting compound consists of fluorescein, rhodamine, and inherently fluorescent molecules that bind to receptors.

11. The process of claim 1 wherein the fluorescence emitting compound consists of a non-steroidal molecule.

12. The process of claim 11 wherein the non-steroidal molecule consists of a peptide derived from transcriptional coregulator proteins that bind to specific sites on receptors.

13. The process of claim 1 wherein the fluorescence emitting compound is a compound selected from the group consisting of AF-1 domain and DNA binding domain.

14. The process of claim 1 wherein the process is performed in multiwell plates.

15. The process of claim 1 wherein more that one receptor is assayed.
